(19) United States
(12) Patent Application Publication
(10) Pub. No.: US 2026/0069619 A1
(43) Pub. Date: Mar. 12, 2026

(54) Treatment of Dry Eye Disease Using Mitochondrially Targeted Antioxidants

(71) Applicant: MITOTECH S.A., Vallee (LU)

(72) Inventors: Maxim SKULACHEV, Moscow (RU); Anton PETROV, Woodland Hills, CA (US)

(21) Appl. No.: 18/872,454

(22) PCT Filed: Jun. 6, 2022
(86) PCT No.: PCT/IB2022/000316

§ 371 Date: Dec. 6, 2024

(51) Int. Cl. A61K 31/66; A61K 31/352; A61K 31/4375; A61K 31/4745; A61P 27/02

(52) U.S. Cl. CPC A61K 31/66; A61K 31/352; A61K 31/4375; A61K 31/4745; A61P 27/02

(57) ABSTRACT

Formulations and methods of using mitochondrially targeted antioxidants (SkQ) to improve dry eye conditions in subjects having a tear production of ≥7 mm measured by a Schirmer's test are provided. The methods provide benefits to the population-specific group of subjects but do not provide benefits to subjects having a tear production of <7 mm, as measured by a Schirmer's test.

### BACKGROUND

A diagnosis of dry eye disease by a health care provider does not require specific signs to be measured by the health care provider. A subject with dry eye disease can report various symptoms to the health care provider, for example, low tear production, poor or blurry vision, painful eyes, gritty or sandy sensations, photosensitivity, eye redness, and itching. The various symptoms can be worsened in conditions such as dry, dusty, or smoky environments, by air conditioning, or by reading and computer use. Symptoms can worsen as a day goes on, but some subjects can experience the worst symptoms after first waking in the morning. The diagnosis can be based solely on the subject's symptoms.

The health care provider may evaluate a subject's symptoms by using a questionnaire such as the Ocular Surface Disease Index (OSDI) questionnaire, which lists 12 questions and grades each response on a scale of 0 (“none of the time”) to 4 (“all of the time”). The health care provider may measure signs indicative of dry eye disease and prescribe medications. Various prescription medications include anti-inflammatory drugs to reduce inflammation of the eyelids, and immuno-suppressive drugs, such as cyclosporine or corticosteroids, to suppress inflammation of the cornea. Eye or tear duct implants can be prescribed. Many subjects rely on over-the-counter remedies including artificial tears or eye drops to reduce eye redness, which may introduce more irritation to the eye, while artificial tears do not target the causes of dry eye disease. There is a need for improved methods for treating and preventing signs and symptoms of dry eye disease, while improving both short-term and long-term outcomes for the subject.

### SUMMARY

The present technology provides population-specific formulations and methods for using mitochondrially-targeted antioxidants (SkQ) to aid in the treatment or prevention of signs and symptoms of dry eye disease (DED). The methods disclosed herein are based on unexpected findings in two consecutive clinical studies, VISTA-1 (NCT03764735) and VISTA-2 (NCT04206020) for subject populations having a tear production of ≥7 mm measured by a Schirmer's test prior to the clinical studies. The active drug substance in these studies is an SkQ type mitochondrially targeted antioxidant. The formulation used in the methods includes the SkQ at a concentration of ≥0.5 μg/mL.

The technology can be further summarized by the following list of features.

1. A method to aid in treating a dry eye condition, the method comprising administering to a subject in need thereof a formulation comprising a compound of Formula I:

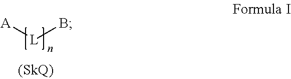

wherein A is a quinone antioxidant having the following structure:

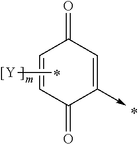

and/or a reduced quinole form thereof; wherein m is an integer from 1 to 3, and each Y is independently selected from the group consisting of C₁₋₆ alkyl and C₁₋₆ alkoxy (—O—C₁₋₆);
wherein L is a linker group, comprising:
   a) a straight or branched hydrocarbon chain which can optionally be substituted by one or more substituents and which can optionally contain one or more double and/or triple bonds; and/or
   b) a natural isoprene chain;
n is an integer from 1 to 40;
B is a targeting group comprising Sk⁺Z⁻; wherein Sk⁺ is a lipophilic cation, and Z⁻ is a pharmaceutically acceptable anion;
wherein the formulation comprises SkQ at a concentration of ≥0.5 μg/mL;
wherein the eye has a tear production of ≥7 mm measured by a Schirmer's test prior to said administering; and
wherein said administering results in an improvement in one or more signs and/or one or more symptoms of dry eye disease.

2. The method of feature 1, wherein the SkQ comprises SkQ1 shown below, in its reduced or oxidized form:

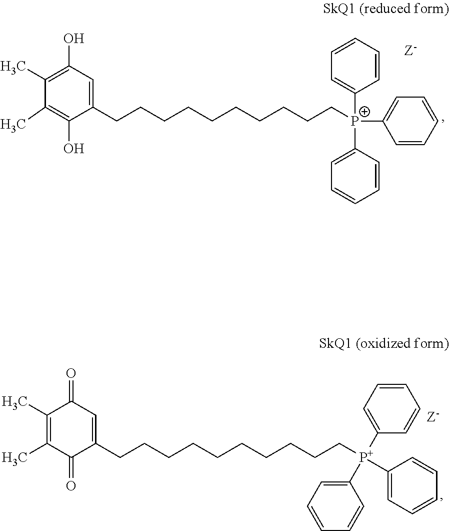 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]

or a combination thereof.

3. The method of any of the preceding features, wherein the formulation is an eye-drop or eye-gel formulation and is applied topically to the eye and/or to one or more adnexa of the eye.

4. The method of any of the preceding features, wherein one or more of said signs of dry eye disease is a grade of corneal staining of >0 in the whole cornea or a portion thereof.

5. The method of any of the preceding features, wherein the one or more symptoms of dry eye disease comprise an index of blurred vision ≥1 or poor vision ≥1, and/or a reduced visual acuity of the eye.

6. The method of any of the preceding features, wherein the formulation is applied topically to the eye, and the formulation contacts one or more adnexa of the eye via a spreading of the formulation on the surface of the eye, an eye blink, an eye movement, or a combination thereof.

7. The method of any of the preceding features, wherein one or more or said signs of dry eye disease is a grade of corneal staining >0 in the whole cornea or a portion thereof, and one of said symptoms of dry eye disease is a reduced visual acuity of the eye.

8. The method of any of the preceding features, further comprising:
administering to the eye a fluorescein stain after a period of time and
measuring a grade of central corneal fluorescein staining; and
repeating the steps of administering the compound of Formula I, administering the fluorescein stain, and measuring a grade of central fluorescein staining, whereby a reduction or a complete clearing in the grade of central corneal fluorescein staining of the eye is achieved.

9. The method of feature 8, wherein the grade of central corneal fluorescein staining is initially >0 and finally=0.

10. The method of any of the preceding features, wherein the subject has a reported index of blurred vision of ≥1 or poor vision of ≥1, and/or a reduced visual acuity of the eye before an administering of the compound of Formula I, and wherein the method provides an improvement in the visual acuity of the eye.

11. The method of feature 10, wherein the improvement in visual acuity is an improvement in a best corrected visual acuity (BCVA) of the eye, and wherein the improvement in BCVA is an improvement of BCVA of about ≥0.3.

12. The method of feature 11, wherein the BCVA is measured as a logarithm of the minimum angle of resolution chart measurement.

13. The method of any of features 11-12, wherein the improvement in BCVA further comprises an improvement in the index of blurred vision and/or the index of poor vision.

14. The method of any of features 10-13, wherein the reported index of blurred vision is a rating on a scale range of 0-4 by the subject of an experience of blurred vision during the week before the administration; wherein 0=none of the time, 1=some of the time, 2=half of the time, 3=most of the time, and 4=all of the time.

15. The method of any of features 10-13, wherein the reported index of poor vision is a rating on a scale range 0-4 by the subject of an experience of poor vision during the week before the administration; wherein 0=none of the time, 1=some of the time, 2=half of the time, 3=most of the time, and 4=all of the time.

16. The method of any of the preceding features, wherein the one or more signs and/or the one or more symptoms of dry eye disease comprise one or more of the following: tear production in the eye of ≥7 mm by a Schirmer's test, a grade of central corneal fluorescein staining of >0, a total fluorescein corneal staining score of ≥2, a total lissamine green conjunctival staining score of ≥2, corneal sensitivity, conjunctival redness, lid margin redness, damage to the cornea, subject eye grittiness, eye itching or discomfort, reduced visual acuity, and subject reported blurred vision, poor vision, eye pain, eye photosensitivity, or eye pain.

17. The method of any of the preceding features, wherein said administration is carried out over a period of about 14 days to about 90 days.

18. The method of any of the preceding features, wherein the one or more signs and/or one or more symptoms of dry eye disease are associated with a condition comprising one or more of the following: eye strain due to reading, driving at night, watching a display screen, or contact lens wear, exposure to an environmental eye irritant, hormone replacement therapy, exposure to the wind or dry air, allergies, eye surgery, a side effect of a medication, thyroid disease, disease of aging, an infection, exposure keratitis, vitamin deficiency, Sjogren's syndrome, and a condition associated with keratoconjunctivitis sicca, keratitis sicca, or an immune system disorder.

19. The method of feature 18, wherein the infection is a bacterial infection, fungal infection, or viral infection.

20. The method of any of the preceding features, wherein the SkQ comprises one or more compounds, in oxidized or reduced form, or a mixture thereof, selected from the group consisting of the following compounds:

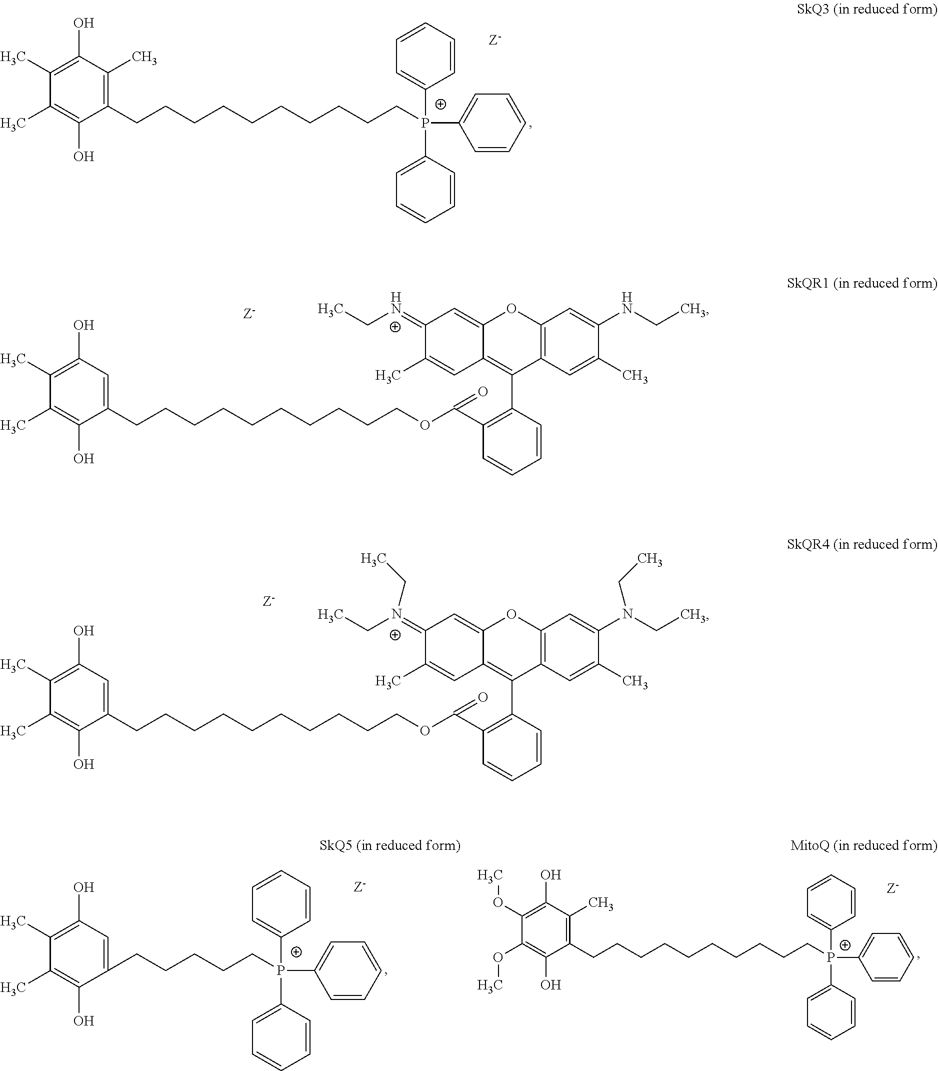

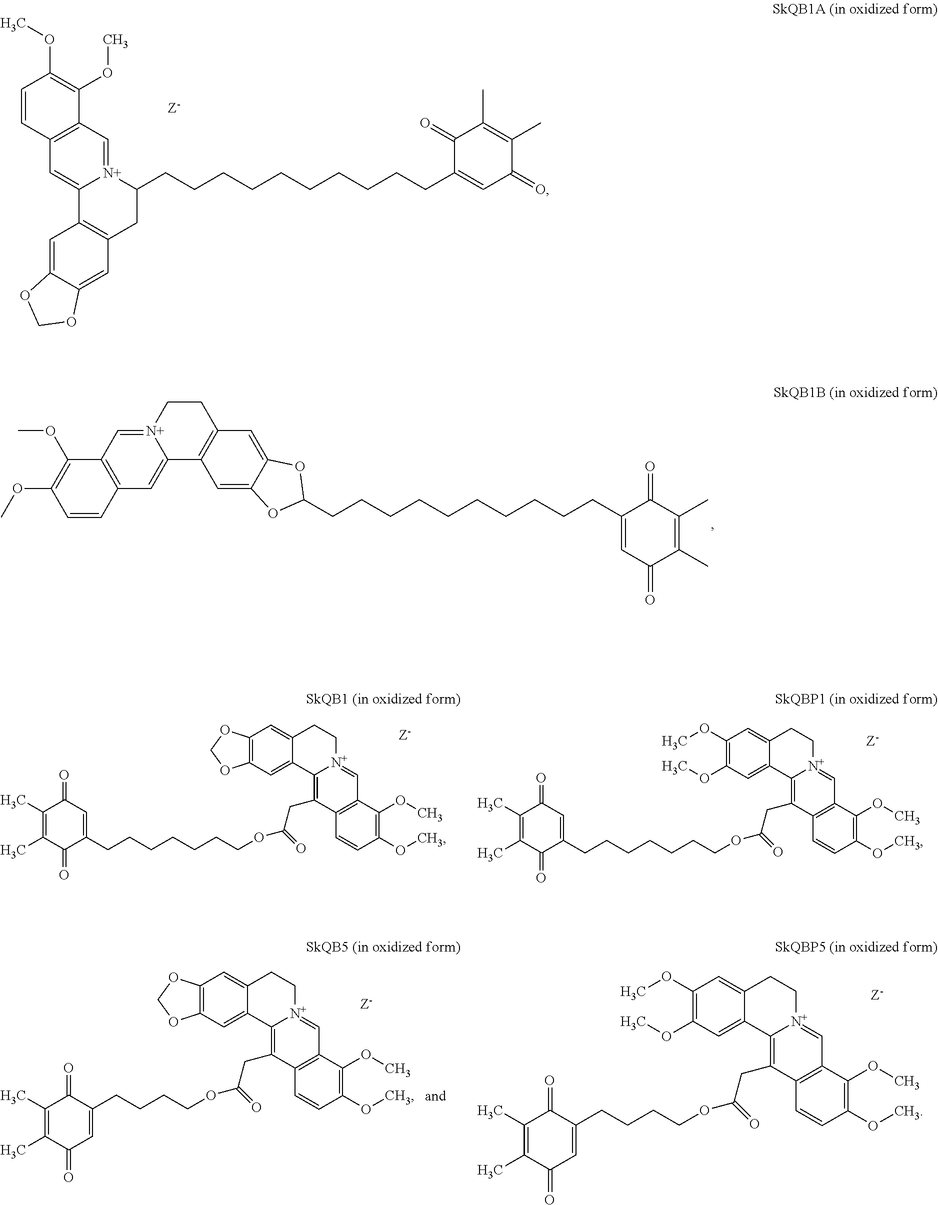

21. The method of any of the preceding features, wherein the pharmaceutically acceptable anion Z⁻ comprises acetate, bromide, camsylate, chloride, formate, fumarate, maleate, mesylate, nitrate, oxalate, phosphate, dihydrogen phosphate dodecahydrate, dihydrogen phosphate dihydrate, sulfate, tartrate, thiocyanate, tosylate, adipate, caprate, caproate, caprylate, dodecylsulfate, glutarate, laurate, oleate, palmitate, sebacate, stearate, undecylenate, or a combination thereof.

22. The method of any of the preceding features, wherein the SkQ optionally comprises a hydrate comprising one or more water molecules associated via a hydrogen bonding and/or an ionic bonding to the A, L, Sk⁺, and/or to Z⁻.

23. The method of any of the preceding features, wherein the administration to the subject is at a dose of at least one drop or at least about 0.05 mL of the formulation administered to the eye and/or to one or more adnexa of the eye at least once every 24 hours.

24. The method of any of the preceding features, wherein the formulation comprises one or more of the following: benzalkonium chloride, hypromellose, sodium chloride, sodium dihydrogen phosphate dihydrate, sodium dihydrogen phosphate dodecahydrate, and water.

25. The method of any of the preceding features, wherein the Schirmer's test score is an unanesthetized Schirmer's test score.

26. The method of any of the preceding features, wherein the one or more symptoms of dry eye disease comprise one or more symptoms reportable by the subject.

27. The method of any of the preceding features, wherein the one or more signs and/or the one or more symptoms of dry eye disease arise from a progressive dry eye disease for longer than about 2 months.

28. The method of feature 27, wherein a damage to the central cornea is indicated by a grade of central corneal fluorescein staining of ≥1.

29. The method of any of the preceding features, wherein the formulation comprises SkQ at a concentration of ≥1 μg/mL.

30. A kit for treating one or more signs and/or one or more symptoms of dry eye disease, the kit comprising:
a dispensing container comprising an eye-drop or eye-gel formulation comprising SkQ at a concentration of ≥0.5 μg/mL; and
instructions for use.

31. The kit of feature 28, wherein the eye-drop or eye-gel formulation comprises SkQ at a concentration of ≥1 μg/mL.

As used herein, the term “dry eye disease” can be used interchangeably with the term “dry eye syndrome”.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### DETAILED DESCRIPTION

The present technology provides population-specific formulations and methods for treatment and prevention of signs or symptoms of DED using a formulation including SkQ at ≥0.5 μg/mL. Two consecutive clinical studies, VISTA-1 and VISTA-2, show unexpected benefits for a specific population of subjects having a pre-treatment (baseline) tear production of ≥7 mm measured by a Schirmer's test. The formulation is administered to this population for a period of time. After the period of time, the formulation provides benefits. Examples of the benefits can include improvement in visual acuity and healing of the central cornea area and of the overall cornea, unexpectedly depending on the baseline measured tear production of the subject.

For example, subjects with a Schirmer's score ≥7 mm at baseline demonstrate statistically significant improvement of a grade of central corneal staining as well as significant improvement in visual acuity compared to vehicle. Surprisingly, subjects with Schirmer's score ≤7 mm at baseline do not demonstrate such improvements compared to vehicle. Using a formulation including SkQ at ≥1 μg/mL, the clinical data demonstrate that presence of tear volume of the level of a Schirmer's score at ≥7 mm is critical for the formulation's efficacy against signs and symptoms of DED.

A Schirmer's test can be used to measure whether an eye produces enough tears to keep it moist. A Schirmer's test can be performed by a health care provider applying certified sterile test strips in the lower eyelid of each eye of the subject for 5 minutes without anesthesia. Certified Schirmer's test strips are also available for purchase without a prescription (over the counter) and may be provided with millimeter calibrations on the strips and instructions for a subject to perform Schirmer's self-testing diagnostics.

Without anesthesia, the Schirmer's test can also evaluate the ability of the eye surface to respond to surface stimulation. A topical eye anesthesia can be used by a health care provider before conducting a Schirmer's test. When an anesthesia is used, the test can evaluate basal tear secretion without taking into the measurement the ability of the eye surface to respond to unanesthetized surface stimulation. The Schirmer's test discussed herein can be an unanesthetized or anesthetized test.

The Schirmer's test strips can be inserted into the lower conjunctival sac at the junction of the lateral and middle thirds, while avoiding touching the cornea, and the length of wetting of the strips (in millimeters) is recorded after 5 minutes. The subject is allowed to blink the eyes normally during the test. After 5 minutes, the test strips are removed and measured to determine how much of each strip has become moist. Schirmer's test results for tear production can be evaluated as follows: a wet segment of >25 mm=hyper lacrimation, 15-24 mm=normal tear production, 10-14 mm=mild level of tear production dysfunction, 5-9 mm=moderate level of tear production dysfunction, and <5 mm=severe level of tear production dysfunction (Brzhesky, et al., 2015).

Mitochondria targeted antioxidants of an SkQ1 type are reported to have some efficacy against pathological DED in a broad population of subjects. Brzhesky, et al., 2015 and Petrov, et al., 2016 show that SkQ1 treatment may lead to corneal protection and improvement of certain signs and symptoms of DED for patients with a wide range of Schirmer's test scores. DED pathogenesis and the progression of signs and symptoms can vary in different cohorts of patients. Brzhesky and Petrov do not suggest a specific method of treatment of DED depending on a population-specific group of subjects, and on a subject's type or stage of the disease.

From Brzhesky and Petrov, pathological DED-related damage of the central area of cornea of DED patients can be reduced by SkQ1 treatment. Unexpectedly, this observation is not confirmed in the Intent to Treat population in the VISTA-1 clinical trial below (Example 1). Central cornea damage healing is an important issue for the treatment of DED because the central region of the cornea is responsible for supporting visual acuity. The cornea acts as the eye's outermost lens. This key role of the cornea in providing visual acuity is recognized by the Food and Drug Administration (FDA).

By analyzing data collected in the VISTA-1 clinical trial and the VISTA-2 clinical trial, it is found that a population-specific group of subjects with a baseline tear production of ≥7 mm, measured by a Schirmer's test, improves in specific signs and symptoms of DED after administration of a formulation including SkQ. For example, the population-specific group surprisingly can have a mild to moderate level of tear production. In the population-specific group, subjects with a progressed or long-term DED can have excessive tears in the dry eye, which may arise due to excessive reflex tearing in a response to corneal surface disease from the DED (Shen, et al., 2016; Messmer, 2015). Using a Schirmer's test for measurement, the baseline tear production can be ≥7 mm, or in a range from ≥7 mm to about ≤50 mm, in a range from ≥7 mm to about ≤40 mm, in a range from ≥7 mm to about ≤30 mm, in a range from ≥7 mm to about ≤25 mm, in a range from ≥7 mm to about ≤20 mm, in a range from ≥7 mm to about ≤15 mm, in a range from ≥7 mm to about ≤14 mm, in a range from ≥7 mm to about ≤13 mm, in a range from ≥7 mm to about ≤12 mm, in a range from ≥7 mm to about ≤11 mm, or in a range from ≥7 mm to about ≤10 mm.

For the population-specific subjects to gain the benefits of the methods disclosed herein, administration of a formulation including SkQ to an eye of the subject must be for a period of time of the administration or for an initial period of time of the administration. The initial period of time of the administration can be followed by one or more periods of time of the administration. The period of time can be in a range from 14 days to about 120 days, in a range from about 21 days to about 100 days, in a range from about 30 days to about 90 days, in a range from about 30 days to about 60 days, in a range from about 30 days to about 40 days, or in a range from about 30 days to about 35 days.

Studies of SkQ1 show a no observed adverse effect level (NOAEL) for ocular toxicity of 4.5 μg per eye per day (4.5 μg/eye/day). Mild ocular toxicity of SkQ1 is observed at 45 μg/eye/day (Petrov, et al., 2016). One drop of an aqueous formulation contains about 0.05 mL. One drop of a gel formulation can contain about 0.05 mL to about 0.1 mL. With a 0.1 mL dose at a high concentration (0.1 mL×1.55 μg/mL, bis in die=0.31 μg/eye/day), the amount of SkQ1 instilled in an eye falls within a safety margin of 10 for systemic exposure observed at the NOAEL for ocular toxicity, (i.e., ≤4.5 μg/eye/day). The methods disclosed herein can include administration of a formulation including SkQ at a concentration in a range from ≥0.5 μg/mL to about ≤22.5 μg/mL, in a range from ≥0.5 μg/mL to about ≤20.0 μg/mL, in a range from ≥0.5 μg/mL to about ≤15.0 μg/mL, in a range from ≥0.5 μg/mL to about ≤10.0 μg/mL, in a range from ≥0.5 μg/mL to about ≤7.5 μg/mL, in a range from ≥0.5 μg/mL to about ≤5.0 μg/mL, in a range from ≥0.5 μg/mL to about ≤2.5 μg/mL, or in a range from ≥0.5 μg/mL to about ≤2.0 μg/mL. In another example, the methods disclosed herein can include administration of a formulation including SkQ at a concentration in a range from ≥1 μg/mL to about <22.5 μg/mL, in a range from ≥1 μg/mL to about ≤20.0 μg/mL, in a range from ≥1 μg/mL to about ≤15.0 μg/mL, in a range from ≥1 μg/mL to about ≤10.0 μg/mL, in a range from ≥1 μg/mL to about ≤7.5 μg/mL, in a range from ≥1 μg/mL to about ≤5.0 μg/mL, in a range from ≥1 μg/mL to about ≤2.5 μg/mL, or in a range from ≥1 μg/mL to about ≤2.0 μg/mL.

Using a topical formulation including SkQ in a range from ≥1 μg/mL to about ≤5.0, administration of the formulation can be carried out once per every two days per eye, once per day per eye, bis in die (BID), three times per eye per day, four times per day per eye, or five times per day per eye. With a formulation including higher concentrations of SkQ, administration of the formulation can be once per every two days per eye, once per day per eye, or BID.

The formulation can be applied topically to the eye and/or to one or more adnexa of the eye. Examples of the adnexa include orbits, extraocular muscles, eyelids, a portion of the lacrimal system, optic nerves, or a combination thereof.

The formulation can be administered systemically (or non-topically). The formulation can be administered intradermal or subdermal as a liquid/gel formulation at higher SkQ concentrations. For example, intra/sub dermal administration can be using a formulation with an osmolarity in the range from about 200 mOsm/L to about 600 mOsm/L, in the range from about 250 mOsm/L to about 450 mOsm/L, or in the range from about 275 mOsm/L to about 325 mOsm/L with no adverse irritation. The formulation can be administered with a carrier formulation, for example, a transdermal patch or an eye carrier, optionally including polymers, adhesives, coatings, or inserts.

The formulation can be administered systemically in other examples by various methods, for example, single injection, daily injections, intravenous (IV) drip, and self-injections. An IV injection formulation is isotonic (e.g., osmolarity/osmolality adjusted with a suitable salt), and pH is monitored as further described below. The formulation can be administered systemically in the form of a nasal spray formulation or in an oral formulation or drug-in-capsule (DC), for example, an oral formulation can be made using one or more excipients selected from milk acid or lactic acid, propylene glycol, and purified water.

The formulation of SkQ can be a formulation of SkQ including water as a solvent for the SkQ. The formulation can be individually packaged in sterile one-time use dispenser packaging. The formulation can be provided in a multi-use dispensing container securing a formulation of an eye-drop or an eye-gel. The formulation of SkQ including water can be sterilized, for example, by ionizing radiation, filtration, heat or microwaves. The formulation of SkQ can include excipients or additional ingredients, for example, hypromellose, sodium chloride, sodium dihydrogen phosphate dihydrate, sodium dihydrogen phosphate dodecahydrate, osmolarity adjusters, and pH buffers.

An eye-drop or eye-gel formulation of SkQ can include preservatives, for example, benzalkonium chloride or polyquaternium-1. The formulation of SkQ can include one or more gelling agents, for example, methyl cellulose, hydroxyethyl cellulose, hydroxypropyl methylcellulose, sodium carboxymethyl cellulose, alginate or sodium alginate, poloxamer, carbomer polymer, and thiolated chitosan. The one or more gelling agents can be formulated as a flowing (non-viscous) solution that forms a gel (viscous solution) only after contacting an eye, for example, by adding sodium chloride to the gelling agent or by lowering the hydroxypropyl molar substitution of a gelling agent. In this example, the temperature of an eye can be utilized to induce gelling of a formulation.

The eye-drop or eye-gel formulation of SkQ can be adjusted to a pH value in a range from about 3.5 to about 6.9, in a range from about 4 to about 6.9, in a range from about 5 to about 6.9, in a range from about 5.5 to about 6.9, or in a range from about 6 to about 6.9. The formulation of SkQ can include osmolarity adjusting agents (e.g., a sugar or NaCl). Osmolarity can be measured, for example, by freezing point osmometry. Normal tear osmolarity can be about 289 mOsm/L, but this value can be changed in subjects with signs or symptoms of DED. The formulation can be adjusted to an osmolarity in a range from about 50 mOsm/L to about 1000 mOsm/L, in a range from about 100 mOsm/L to about 700 mOsm/L, in a range from about 150 mOsm/L to about 600 mOsm/L, or in a range from about 200 mOsm/L to about 500 mOsm/L.

The methods disclosed herein can provide one or more improvements in a grade of corneal staining in the whole cornea or in a portion (zone) of the cornea. A fluorescent or visual staining technique can be used to grade five different zones of the cornea (central, superior, inferior, nasal, and temporal) for damage on a scale with a range of 0-3 (0=no damage, 3=most damage) or a scale with a range of 0-4 (0=no damage, 4=most damage). The grade of 3 or 4 (most damage) can be a confluent or severe staining of the zone. The grade of whole cornea damage can be calculated as a total damage score of the sum of the zone scores. In this example, the maximum possible grade of whole corneal damage score is 15 or 20, respectively. Alternatively, three different zones of the cornea (central, superior, inferior) can be graded for damage on a scale with a range of 0-3 (0=no damage, 3=most damage) or a scale with a range of 0-4 (0=no damage, 4=most damage). In this example, the maximum possible grade of whole corneal damage score is 9 or 12, respectively. A total score of 2 or higher, 3 or higher or 4 or higher can be considered abnormal.

The dye used for staining can be, for example, fluorescein, lissamine green dye, or rose bengal dye (Begley, et al., 2019). The National Eye Institute/Industry Workshop Scale (NEI scale) can be utilized. The NEI scale is a 4-point NEI scale in the range from 0=no staining present to 3=severe staining.

A grade of the central corneal staining can be assigned on a scale of 0-3 or a scale of 0-4, where grade 0=none and grade 4=severe or confluent staining. A grade of conjunctival staining can be calculated by the sum of the grade of the temporal zone and the grade of the nasal zone of the cornea. During a grading of whole cornea damage or of central corneal staining, an observation of an acute severe damage, (e.g., punctate epithelial erosions), can be immediately assigned a grade of 3 or 4.

The methods herein can provide a mean reduction in the grade of central corneal fluorescein staining for the selected populations of greater than about 0.1, greater than about 0.2, greater than about 0.3, greater than about 0.4, or optionally in a range from about 0.1 to about 1.

A health care provider can measure a subject's blink rate as a sign of DED. Blinks can be further categorized into different types of blinks (e.g., spontaneous, incomplete, with or without reading). In an example of a sum of all total blinks, the average blink rate can be about two times higher in dry eyes than in non-dry eyes, or about 28 blinks/minute in a dry eye and about 13 blinks/minute in a normal eye, or about ≥20 blinks/minute in a dry eye and in a range from about 10-15 blinks/minute in a normal eye. A range between about 15 and 20 blinks/minute can be further investigated.

A health care provider can assess conjunctival redness of a subject's eye by using a scale including 3-5 points with the highest score being the most severe. Lid margin redness can be assessed by using a scale including 3 or 4 points with the highest score being the most severe.

The Schirmer's test discussed herein or any of the signs and/or symptoms discussed herein can be measured with or without implementation of controlled measurement conditions.

Ocular discomfort scores can be subjectively graded by a subject according to a discomfort scale (0-4, with 0=no discomfort and 4=constant discomfort) for each eye. Similar scales can be used, for example, in a range of 0-3, where 0=no discomfort and 3=constant or severe discomfort.

Tear breakup time (TBUT) is a test used to assess for signs of evaporative DED. To measure TBUT, a dye (e.g., fluorescein) is instilled into the subject's tear film and the subject is asked not to blink while the tear film is observed under a broad beam of cobalt blue illumination. The TBUT is recorded as the number of seconds that elapse between the last blink and the appearance of the first dry spot in the tear film. A TBUT under about 10 seconds is considered an abnormal sign indicative of DED.

Visual acuity can be measured by a health care provider to provide a sign of DED. The visual acuity can be a best corrected visual acuity (BCVA) of an eye or of both eyes. A logarithm of the minimum angle of resolution (LogMAR) chart such, for example, a Bailey-Lovie chart or an Early Treatment Diabetic Retinopathy Study chart can be utilized to measure BCVA. The number of letters read correctly by a subject can be converted to logMAR scores.

A Snellen chart or a Golovin-Sivtsev table can be utilized to measure BCVA. Using a Golovin-Sivtsev table, a score of 1.0=good vision (equivalent to 20/20 on the Snellen chart) and approaching 0.0=poor vision (e.g., 0.1=20/200 on the Snellen chart). For example, visual acuity can be determined using Golovin-Sivtsev tables illuminated at 600-1000 lux at a five-meter distance. In another example, a Snellen score of 6/6 (20/20), indicating that a subject can resolve details as small as 1 minute of visual angle, corresponds to a LogMAR of 0 (e.g., the base-10 logarithm of 1 is 0). A Snellen score of 6/12 (20/40), indicating a subject can resolve details as small as 2 minutes of visual angle, corresponds to a LogMAR of 0.3 (e.g., the base-10 logarithm of 2 is near-approximately 0.3). Electronic visual acuity testing or computerized visual acuity testing can be applied instead of or in addition to the charts and tables.

Dilated fundoscopy examinations or dilated-pupil examinations (fundus) can be conducted by a health care provider. The fundus examinations can include examinations of the vitreous, retina, macula, choroid, and optic nerve. Each examination can be recorded as normal, abnormal non-clinically significant (NCS), or abnormal clinically significant (CS). A grading scale (e.g., 0-3 or 0-4) as described above can be utilized.

A grade of symptoms of dry eye disease can be calculated by a sum of individual symptoms, situations or conditions, and problems reported by a subject. A questionnaire or an interview by a health care provider can be utilized to receive the reports of symptoms from the subject.

A subject can report one or more symptoms of dry eye disease. Examples of symptoms are eyes that are sensitive to light (photosensitivity), eyes that feel dry or gritty, painful, uncomfortable, or sore eyes, blurred or poor vision, low tear production, eye redness, and itching. Each symptom can be individually reported or rated on a numerical index by the subject or during an interview by a health care provider. The index can, for example, be on a scale of 0-4, wherein 0 indicates none of the time, 1 indicates some of the time, 2 indicates half of the time, 3 indicates most of the time, and 4 indicates all of the time.

A subject can report feeling uncomfortable eyes in situations or conditions. Examples of conditions are windy conditions, low humidity or air-conditioned conditions. Each condition can be individually reported or rated on a numerical index by the subject indicating degree of discomfort. The index can, for example, be on a scale of 0-4, wherein 0 indicates none of the time, 1 indicates some of the time, 2 indicates half of the time, 3 indicates most of the time, and 4 indicates all of the time.

A subject can report one or more problems associated with symptoms of dry eye disease. Examples of problems are problems with reading, driving at night, working with a computer screen, or watching a movie. Each problem can be individually reported or rated on a numerical index by the subject. The index can, for example, be on a scale of 0-4, wherein 0 indicates none of the time, 1 indicates some of the time, 2 indicates half of the time, 3 indicates most of the time, and 4 indicates all of the time.

A diagnosis of DED can be based solely on the subject's report of one or more symptoms, one or more situations or conditions causing discomfort, or one or more problems. A diagnosis of DED can be based solely on signs of dry eye disease including one or more signs provided by a diagnosis of a health care provider. The diagnosis of DED can include an estimate of how long a subject has had DED. For example, central cornea damage can indicate a subject has had DED for a long-term pathogenesis and/or progression of DED.

A percentage can be calculated by the composite or total possible sum of the symptoms, situations or conditions, and problems reported by a subject. A severe dry eye disease can be about ≥50% of the total possible sum. A moderate dry eye disease can be in the range from about ≥30% to ≤50% of the total possible sum. A mild dry eye disease can be about ≥10% to ≤30%. Normal eyes can be ≤10% of the possible sum. Each individual score can be assigned a weight. For example, the OSDI is a questionnaire or interview that has been validated to discriminate between normal, mild to moderate, and severe dry eye disease using a total disease severity score (Bakkar, et al., 2021). The use of a survey or the OSDI has been demonstrated in numerous clinical trials and in-office diagnoses.

Oxidative stress can be involved in a variety of diseases including ocular conditions such as macular degeneration, uveitis, cataracts, corneal and ocular surface inflammation, and DED. Paradoxically, subjects with a progressed or long-term DED can complain of excessive tears in the dry eye, which may arise due to excessive reflex tearing in a response to corneal surface disease from the DED (Shen, et al., 2016; Messmer, 2015). The population-specific methods disclosed herein can apply to a group of subjects with a Schirmer's score ≥7 mm at baseline at least partially arising due to an increased tearing from the pathogenesis and the progression of long-term or progressed DED. Application of the method to the specific group of subjects with a Schirmer's score >7 mm at baseline can also be at least partially due to SkQ acting through reinforcing natural antioxidant composition of the tear, possibly depleted by DED.

The Schirmer's test score ≥7 mm at baseline can be combined with an estimate of progression of DED in the population-specific group. The methods can be applied to subjects having a Schirmer's test score ≥7 mm at baseline and one or more of a grade of corneal staining in the whole cornea or in a portion of the cornea ≥1, an abnormal blink rate, conjunctival or lid margin redness, abnormal ocular discomfort score, TBUT, or visual acuity. In an example, the population-specific methods can be applied to progressive DED (e.g., damage to the central cornea ≥1 grade) and a Schirmer's test score ≥7 mm at baseline at least partially arising from DED for longer than about 2 weeks, longer than about 1 month, longer than about 2 months, longer than about 3 months, longer than about 6 months, longer than about 1 year, or longer than about 2 years (Shen, et al., 2016; Messmer, 2015). The role of SkQ in the methods can address the oxidative stress or damage to the central cornea in long-term DED.

The methods disclosed herein can be used with one or more other concomitant therapies. The one or more other concomitant therapies can be associated with DED or with one or more different ailments or conditions. The DED can be associated with exposure to one or more of a variety of conditions, for example, keratoconjunctivitis sicca, or keratitis sicca. The DED can be a multifactorial disease of the eye surface.

The aging process, which typically results in decreased tear production and flow, and certain hormonal changes (e.g., during menopause) are major contributors to DED. Oxidative stress is involved in the natural aging process. Environmental and lifestyle contributors to DED are numerous and can include: side effects of stress related ailments, air quality index near a city, exposure to low humidity outdoors or indoors, tobacco smoke, wind, sun (UV-light or heat) exposure, ozone, dusty work environments, side effects of certain medications, such as antihistamines, antidepressants, and beta-blockers; certain medical conditions, including other ocular conditions (e.g., blepharitis, allergic conjunctivitis), rheumatoid arthritis, Sjogren's syndrome, cataract surgery, or contact dermatitis. Contact lens use and activities that decrease blinking, such as prolonged computer use or reading, can contribute to DED.

### EXAMPLES

### Example 1. VISTA-1 Clinical Study

The VISTA-1 clinical study (NCT03764735) evaluated ophthalmic solutions with different concentrations of SkQ1 for various aspects of safety and effectiveness compared to placebo (vehicle) for treatment of various signs and symptoms of DED. The vehicle was an artificial tear treatment used as a control.

The VISTA-1 study reported change of level of corneal fluorescein staining from baseline to day 57. The level of corneal fluorescein scale ranged from 0 to 4, where level 0=none and level 4=severe. VISTA-1 reported a change in symptoms such as subject-reported grittiness from baseline to day 57, where the scale ranged from 0 to 5 for each symptom, where 0=none and 5=worst. Study subjects were carefully monitored for any adverse events. Example aspects of the VISTA-1 study design are summarized in Table 1 below.

TABLE 1
Example Aspects of VISTA-1 Study Design.

| Aspect: | Description: |
|---|---|
| Structure | Multi-center, double-masked, randomized, placebo- |
| | controlled study |
| Duration | An individual subject's participation is estimated |
| | to be approximately 9 weeks (63 days) |
| Controls | Placebo SkQ1 ophthalmic solution (vehicle of SkQ1 |
| | ophthalmic solution) |
| Dosage/Dose | Subjects eligible to be randomized will receive |
| Regimen | one of the following treatments to be |
| | administered bilaterally 1-2 drops bis in die |
| | (BID) for 56 days (from visit 2 to visit 5): |
| | 1) 0.155 μg/mL SkQ1 ophthalmic solution |
| | 2) 1.55 μg/mL SkQ1 ophthalmic solution |
| | 3) Placebo SkQ1 ophthalmic solution (vehicle) |
| Measures Taken | VISTA-1 was a double-masked study |
| to Reduce Bias | |

Study Population Characteristics:

| | |
|---|---|
| Number of | Subjects screened to enroll approximately 444 |
| Subjects: | (148 low dose: 148 high dose: 148 placebo) |
| | subjects |
| Condition/ | Dry eye syndrome |
| Disease | |

### VISTA-1 Study Results

The mean change from baseline in corneal fluorescein staining values in the central region at for all treatment groups were −0.31 in the 0.155 μg/mL SkQ ophthalmic solution group, −0.20 in the 1.55 μg/mL SkQ6 ophthalmic solution group, and −0.26 in the placebo group. The mean differences between active and placebo were not statistically significant, thus indicating that SkQ1 ophthalmic solution had no statistically significant beneficial treatment effect on the central fluorescein staining sign of dry eye. Table 2 presents a summary of change from baseline in central corneal fluorescein staining (after 57 days) in the mITT population in VISTA-1.

TABLE 2
Change From Baseline in Central Corneal Fluorescein Staining.

| Statistical | 0.155 μg/mL | 1.55 μg/mL | |
|---|---|---|---|
| Measurement | SkQ1 | SkQ1 | Placebo |
| N | 149 | 151 | 151 |
| Mean (SD) | −0.30 (0.816) | −0.20 (0.763) | −0.28 (0.773) |
| P value, Paired t-test | <0.0001 | 0.0020 | <0.0001 |

Active - Placebo
ANCOVA model

| | | | |
|---|---|---|---|
| LS Mean (SE) | −0.31 (0.059) | −0.20 (0.058) | −0.26 (0.058) |
| P value | 0.5799 | 0.4943 | — |

Two-sample t-test

| | | | |
|---|---|---|---|
| Difference in Means | −0.02 | 0.08 | — |
| P value | 0.8431 | 0.3608 | — |

### Example 2. VISTA-2 Clinical Study

The VISTA-2 (NCT04206020) clinical study reported results of a dose of 1.55 μg/mL SkQ1 compared to placebo (vehicle). VISTA-2 reported a measured change of ocular discomfort scale. The scale ranged from 0 to 4 with 0=no discomfort and 4=constant discomfort from baseline to Day 57. VISTA-2 reported a measured change of level of conjunctival fluorescein staining (sum of temporal and nasal regions). The scale in each region ranged from 0 to 4, with 0=no staining and 4=confluent staining from baseline to day 57. Study subjects were carefully monitored for any adverse events. Example aspects of the VISTA-2 study design are summarized in Table 3 below.

TABLE 3
Example Aspects of VISTA-2 Study Design.

| Aspect: | Description: |
|---|---|
| Structure | Multi-center, double-masked, randomized, placebo- |
| | controlled study |
| Duration | An individual subject's participation is estimated |
| | to be approximately 9 weeks (63 days) |
| Controls | Placebo SkQ1 ophthalmic solution (vehicle of SkQ1 |
| | ophthalmic solution) |
| Dosage/Dose | Subjects eligible to be randomized will receive |
| Regimen | one of the following treatments to be administered |
| | bilaterally as 1-2 drops BID for 56 days (from |
| | visit 2 to visit 5): |
| | 1) 1.55 μg/mL SkQ1 ophthalmic solution |
| | 2) Placebo SkQ1 ophthalmic solution (vehicle) |
| Measures | VISTA-2 was a double-masked study |
| Taken to | |
| Reduce Bias | |

Study Population Characteristics:

| | |
|---|---|
| Number of | Subjects screened to enroll approximately 600 |
| Subjects | (300 active: 300 placebo) subjects |
| Condition/ | Dry eye syndrome |
| Disease | |

### Example 3. Combined Analyses of VISTA-1 and VISTA-2 Clinical Studies

In addition to the reported data, observed data collected during the VISTA-1 and VISTA-2 clinical studies were analyzed in an ITT subgroup with an unanesthetized Schirmer's Test ≥7 mm at baseline (initial evaluation).

In both VISTA-1 and VISTA-2, post-hoc analyses were conducted on observed data for the ITT subgroup including subjects with an unanesthetized Schirmer's test score of ≥7 mm at baseline, with observed data only to evaluate change from baseline in central fluorescein staining. Table 4 summarizes the results for subjects with unanesthetized Schirmer's test score of >7 mm at baseline. In VISTA-1, significant improvement was seen in change from baseline in central fluorescein staining with 1.55 μg/ml SkQ1 (p_{ANCOVA}=0.023). In VISTA-2, with increased sample size, significant improvement was confirmed in this subgroup (p_{ANCOVA}=0.0168). In Table 4, the change from baseline in central fluorescein staining for the ITT subgroup with unanesthetized Schirmer's test score of ≥7 mm at baseline is provided with observed data only.

TABLE 4
Change From Baseline in Central Fluorescein
Staining - ITT Subgroup.

| VISTA-1 | | VISTA-2 | |
|---|---|---|---|
| 1.55 | | 1.55 | |
| μg/mL | Placebo | μg/mL | Placebo |
| SkQ1 | SkQ1 | SkQ1 | SkQ1 |
| (N = 47) | (N = 44) | (N = 80) | (N = 90) |

Change from Baseline

| | | | | |
|---|---|---|---|---|
| LS Mean | −0.46 | −0.12 | −0.43 | −0.18 |
| (SE) | (0.104) | (0.107) | (0.075) | (0.070) |
| p-value vs. | 0.0230 | — | 0.0168 | — |
| Vehicle | | | | |

A similar subgroup, as described above, was also analyzed in post-hoc analyses for both VISTA-1 and VISTA-2 to evaluate the proportion of subjects with clearing of corneal fluorescein staining in the central region. Table 5 summarizes the results for subjects with unanesthetized Schirmer's test ≥7 mm at baseline. In VISTA-1, numerical improvement was observed in these subjects (p_{Pearson chi-squared}=0.0336). In VISTA-2, with increased sample size, significant improvement was confirmed (p_{Logistic regression}=0.0049, p_{Pearson chi-squared}=0.0033, p_{Fisher's exact test}=0.0037). In Table 5, the proportion of study subjects with clearing of corneal fluorescein staining in the central corneal region, for the ITT Subgroup with unanesthetized Schirmer's test score of ≥7 mm at baseline is presented, with observed data only.

TABLE 5
Proportion of Subjects with Clearing of Corneal Fluorescein Staining.

| | VISTA-1 | | | | VISTA-2 | | | |
|---|---|---|---|---|---|---|---|---|
| | | | Placebo | | | | Placebo | |
| | 1.55 μg/mL | | SkQ1 | | 1.55 μg/mL | | SkQ1 | |
| | SkQ1 (N = 47) | | (N = 44) | | SkQ1 (N = 80) | | (N = 90) | |
| Total Number of | 46 | | 43 | | 78 | | 90 | |
| Subjects | | | | | | | | |
| Number of Subjects | 7 | (15.2%) | 1 | (2.3%) | 13 | (16.7%) | 3 | (3.3%) |
| without Fluorescein | | | | | | | | |
| Staining in the Central | | | | | | | | |
| Region (=0 Score) | | | | | | | | |
| Number of Subjects | 39 | (84.8%) | 42 | (97.7%) | 65 | (83.3%) | 87 | (96.7%) |

with Fluorescein
Staining in the Central
Region (>0 Score)

1.55 μg/mL SkQ1 - Placebo SkQ

| Logistic Regression | | | | | | |
|---|---|---|---|---|---|---|
| Odds Ratio (95% CI) | 7.57 | (0.89, 64.36) | — | 6.73 | (1.79, 25.41) | — |

| p-value | 0.0639 | — | 0.0049 | — |
|---|---|---|---|---|
| Pearson's Chi-Squared Test | | | | |
| p-value | 0.0336 | — | 0.0033 | — |
| Fisher's Exact Test | | | | |
| p-value | 0.0593 | — | 0.0037 | — |

In VISTA-2, change from baseline in BCVA using a logMAR (BCVA-logMAR) at Visit 3 was evaluated in the ITT subgroup including subjects with Schirmer's test score ≥7 mm, blurred vision [OSDI 4]≥1, and poor vision [OSDI 5]≥1 at baseline.

Table 6 summarizes the results of VISTA-2. Significant improvement was shown in VISTA-2 with p_{ANCOVA}=0.0143. Table 6 summarizes the BCVA-logMAR change from baseline in 1.55 μg/mL SkQ1 vs Placebo for the ITT subgroup including subjects with Schirmer's test score of ≥7 mm, blurred vision [OSDI 4]≥1, and poor vision [OSDI 5]≥1 at baseline.

TABLE 6
BCVA-logMAR Summary.

VISTA-2

| | 1.55 μg/mL SkQ1 | Placebo SkQ1 |
|---|---|---|
| | (N = 43) | (N = 49) |
| LS Mean (SE) | −0.012 (0.0143) | 0.037 (0.0133) |
| p-value vs. Vehicle | 0.0143 | — |

Importantly, a subgroup including subjects with unanesthetized Schirmer's test score of <7 mm at baseline did not show statistically significant improvement (relative to the vehicle) neither for corneal staining nor for visual acuity. Table 7 and Table 8 summarize the lack of efficacy (compared to the vehicle) for subjects with unanesthetized Schirmer's test score of <7 mm at baseline. Table 7 summarizes a change from baseline in central fluorescein staining for the ITT subgroup with unanesthetized Schirmer's test score of <7 mm at baseline with observed data only.

TABLE 7
Change From Baseline in Central Fluorescein Staining.

| VISTA-1 | | VISTA-2 | |
|---|---|---|---|
| 1.55 | | 1.55 | |
| μg/mL | Placebo | μg/mL | Placebo |
| SkQ1 | SkQ1 | SkQ1 | SkQ1 |
| (N = 104) | (N = 107) | (N = 221) | (N = 218) |

Change from Baseline

| | | | | |
|---|---|---|---|---|
| LS Mean | −0.20 | −0.15 | −0.156 | −0.196 |
| (SE) | (0.098) | (0.107) | (0.110) | (0.091) |
| p-value | 0.67 | — | 0.65 | — |
| vs. Vehicle | | | | |

Table 8 summarizes the BCVA-logMAR change from baseline in 1.55 μg/mL SkQ1 vs Placebo for the ITT subgroup including subjects with Schirmer's test score <7 mm, blurred vision [OSDI 4]≥1, and poor vision [OSDI 5]≥1 at baseline

TABLE 8
BCVA-logMAR, Change from Baseline,
1.55 μg/mL SkQ1 vs Placebo.

VISTA-2

| | 1.55 μg/mL SkQ1 | Placebo SkQ1 |
|---|---|---|
| | (N = 129) | (N = 132) |
| LS Mean (SE) | −0.0 (0.0137) | 0.011 (0.0131) |
| p-value vs. Vehicle | 0.31 | — |

Surprisingly, the example benefits demonstrated in Tables 4-6 were not observed in the data presented in Tables 7-8, where the ITT subgroup including subjects with Schirmer's test score <7 mm.

### REFERENCES

OSDI®, Ocular Surface Disease Index®, AbbVie, Allergan plc, Irvine, CA
NCT03764735, (ClinicalTrials.gov Identifier: NCT03764735).
NCT04206020, (ClinicalTrials.gov Identifier: NCT04206020).
FDA, (fda.gov/medical-devices/lasik/what-lasik).
Petrov, A., et al., SkQ1 ophthalmic solution for dry eye treatment: results of a phase 2 safety and efficacy clinical study in the environment and during challenge in the controlled adverse environment model. Advances in therapy, 2016. 33(1): p. 96-115.
Brzheskiy, V. V., et al., Results of a Multicenter, Randomized, Double-Masked, Placebo-Controlled Clinical Study of the Efficacy and Safety of Visomitin Eye Drops in Patients with Dry Eye Syndrome. Adv Ther, 2015. 32(12): p. 1263-79.
Bakkar M M, EI-Sharif A K, Al Qadire M. Validation of the Arabic version of the Ocular Surface Disease Index Questionnaire. Int J Ophthalmol. 2021; 14(10):1595-1601. Published 2021 Oct. 18. doi:10.18240/ijo.2021.10.18.
Begley C, Caffery B, Chalmers R, Situ P, Simpson T, Nelson J D. Review and analysis of grading scales for ocular surface staining. Ocul Surf. 2019 April; 17(2):208-220. doi: 10.1016/j.jtos.2019.01.004. Epub 2019 Jan. 14. PMID: 30654024.
Shen G L, Ng J D, Ma X P. Etiology, diagnosis, management and outcomes of epiphora referrals to an oculoplastic practice. Int J Ophthalmol. 2016; 9(12):1751-1755. Published 2016 Dec. 18. doi:10.18240/ijo.2016.12.08.
Messmer, Elisabeth M. “The pathophysiology, diagnosis, and treatment of dry eye disease.” Deutsches Arzteblatt international vol. 112, 5 (2015): 71-81; quiz 82. doi:10.3238/arztebl.2015.0071.

1. A formulation comprising a compound of Formula I: 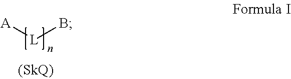
wherein A is a quinone antioxidant having the following structure: 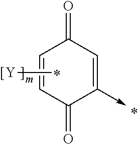
and/or a reduced quinole form thereof; wherein m is an integer from 1 to 3, and each Y is independently selected from the group consisting of C₁₋₆ alkyl and C₁₋₆ alkoxy (—O—C₁₋₆);
wherein L is a linker group, comprising:
a) a straight or branched hydrocarbon chain which can optionally be substituted by one or more substituents and which can optionally contain one or more double and/or triple bonds; and/or
b) a natural isoprene chain;
n is an integer from 1 to 40;
B is a targeting group comprising Sk⁺Z⁻; wherein Sk⁺ is a lipophilic cation, and Z⁻ is a pharmaceutically acceptable anion;
wherein the formulation comprises SkQ at a concentration of ≥0.5 μg/mL; and
wherein the formulation is suitable to aid in treating a subject having a dry eye condition. the subject's eye having a tear production of ≥7 mm measured by a Schirmer's test, and providing an improvement in one or more signs and/or one or more symptoms of dry eye disease.

2. The formulation of claim 1, wherein the SkQ comprises SkQ1 shown below, in its reduced or oxidized form: 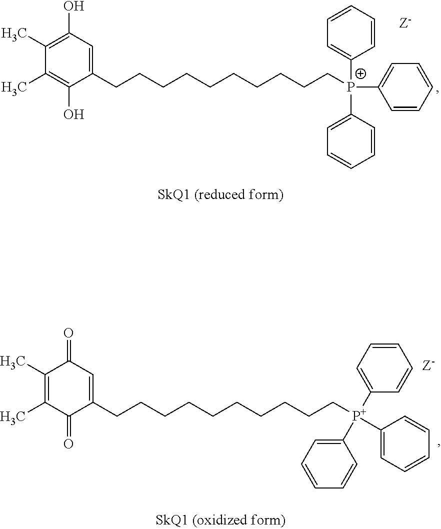 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]
or a combination thereof.

3. The formulation of any of the preceding claims, wherein the formulation is an eye-drop or eye-gel formulation and is suitable to be applied topically to the eye and/or to one or more adnexa of the eye.

4. The formulation of any of the preceding claims, wherein the one or more signs of dry eye disease comprise a grade of corneal staining of >0 in the whole cornea or a portion thereof.

5. The formulation of any of the preceding claims, wherein the one or more symptoms of dry eye disease comprise an index of blurred vision ≥1 or poor vision ≥1, and/or a reduced visual acuity of the eye.

6. The formulation of any of the preceding claims, wherein the formulation is suitable to be applied topically to the eye, and the formulation is suitable to contact one or more adnexa of the eye via a spreading of the formulation on the surface of the eye, an eye blink, an eye movement, or a combination thereof.

7. The formulation of any of the preceding claims, wherein the one or more or said signs of dry eye disease comprise a grade of corneal staining >0 in the whole cornea or a portion thereof, and the one or more symptoms of dry eye disease comprise a reduced visual acuity of the eye.

8. The formulation of any of the preceding claims, wherein the formulation is suitable for use in a method comprising the steps of:
(a) applying a fluorescein stain to the eye;
(b) measuring a grade of central corneal fluorescein staining;
(c) repeatedly treating the eye with the formulation after steps (a) and (b);
thereby providing a reduction or a complete clearing in the grade of central corneal fluorescein staining of the eye.

9. The formulation of claim 8, wherein the grade of central corneal fluorescein staining is initially >0 and finally=0.

10. The formulation of any of the preceding claims, wherein the subject has a reported index of blurred vision of ≥1 or poor vision of ≥1, and/or a reduced visual acuity of the eye before a treatment with the formulation, and wherein the formulation is suitable to provide an improvement in the visual acuity of the eye after the treatment.

11. The formulation of claim 10, wherein the improvement in visual acuity is an improvement in a best corrected visual acuity (BCVA) of the eye, and wherein the improvement in BCVA is an improvement of BCVA of about ≥0.3.

12. The formulation of claim 11, wherein the BCVA is measured as a logarithm of the minimum angle of resolution chart measurement.

13. The formulation of claim 11 or 12, wherein the improvement in BCVA further comprises an improvement in the index of blurred vision and/or the index of poor vision.

14. The formulation of any of claims 10-13, wherein the reported index of blurred vision is a rating on a scale range of 0-4 by the subject of an experience of blurred vision during the week before a treatment with the formulation; wherein 0=none of the time, 1=some of the time, 2=half of the time, 3=most of the time, and 4=all of the time.

15. The formulation of any of claims 10-13, wherein the reported index of poor vision is a rating on a scale range 0-4 by the subject of an experience of poor vision during the week before a treatment of the subject; wherein 0=none of the time, 1=some of the time, 2=half of the time, 3=most of the time, and 4=all of the time.

16. The formulation of any of the preceding claims, wherein the one or more signs and/or the one or more symptoms of dry eye disease comprise one or more of the following: tear production in the eye of ≥7 mm by a Schirmer's test, a grade of central corneal fluorescein staining of >0, a total fluorescein corneal staining score of ≥2, a total lissamine green conjunctival staining score of ≥2, corneal sensitivity, conjunctival redness, lid margin redness, damage to the cornea, subject eye grittiness, eye itching or discomfort, reduced visual acuity, and subject reported blurred vision, poor vision, eye pain, eye photosensitivity, or eye pain.

17. The formulation of any of the preceding claims, wherein the formulation is suitable for treating the subject's eye over a period of about 14 days to about 90 days.

18. The formulation of any of the preceding claims, wherein the one or more signs and/or one or more symptoms of dry eye disease are associated with a condition comprising one or more of the following: eye strain due to reading, driving at night, watching a display screen, or contact lens wear, exposure to an environmental eye irritant, hormone replacement therapy, exposure to the wind or dry air, allergies, eye surgery, a side effect of a medication, thyroid disease, disease of aging, an infection, exposure keratitis, vitamin deficiency, Sjogren's syndrome, and a condition associated with keratoconjunctivitis sicca, keratitis sicca, or an immune system disorder.

19. The formulation of claim 18, wherein the infection is a bacterial infection, fungal infection, or viral infection.

20. The formulation of any of the preceding claims, wherein the SkQ comprises one or more compounds, in oxidized or reduced form, or a mixture thereof, selected from the group consisting of the following compounds:

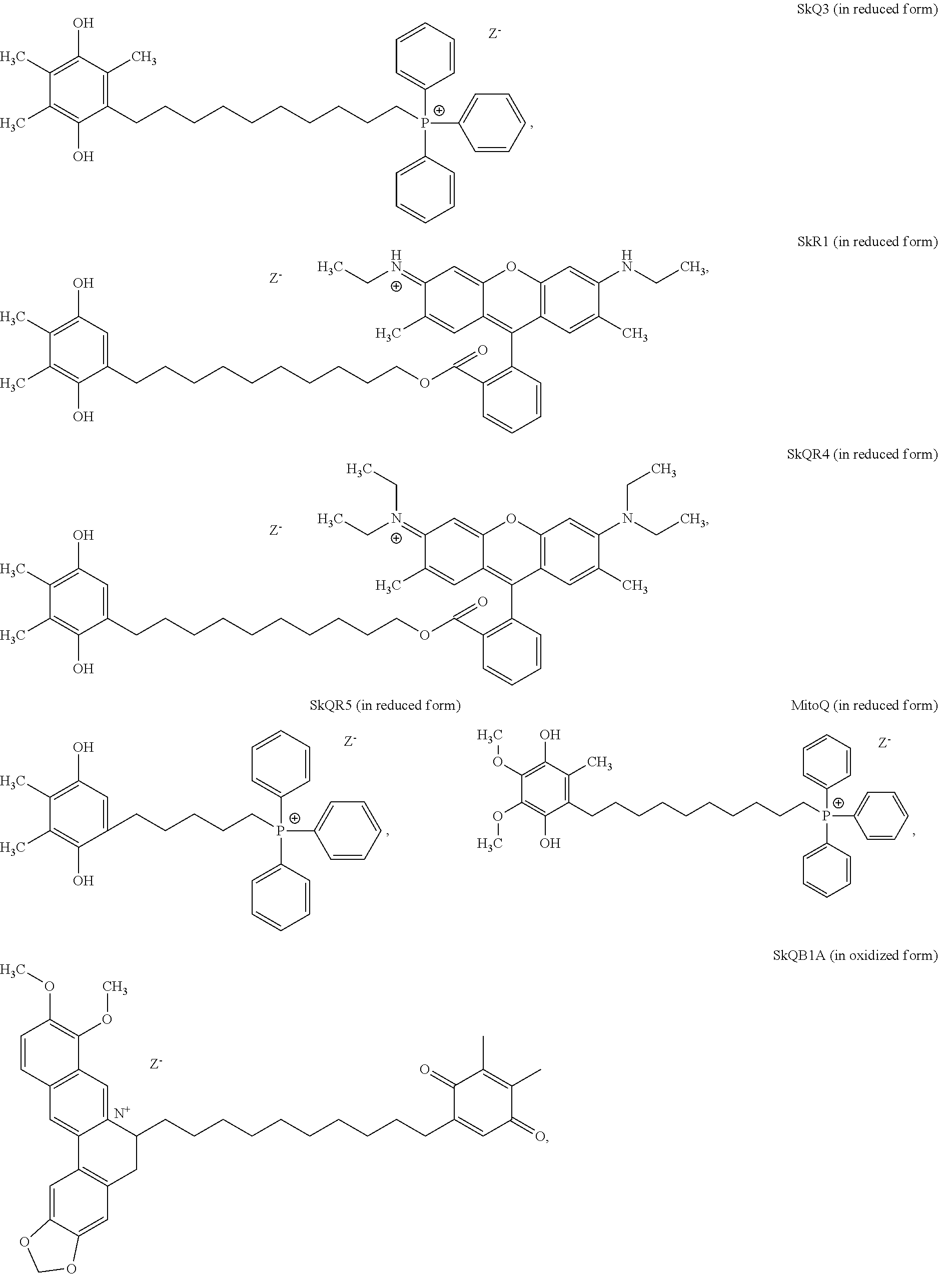

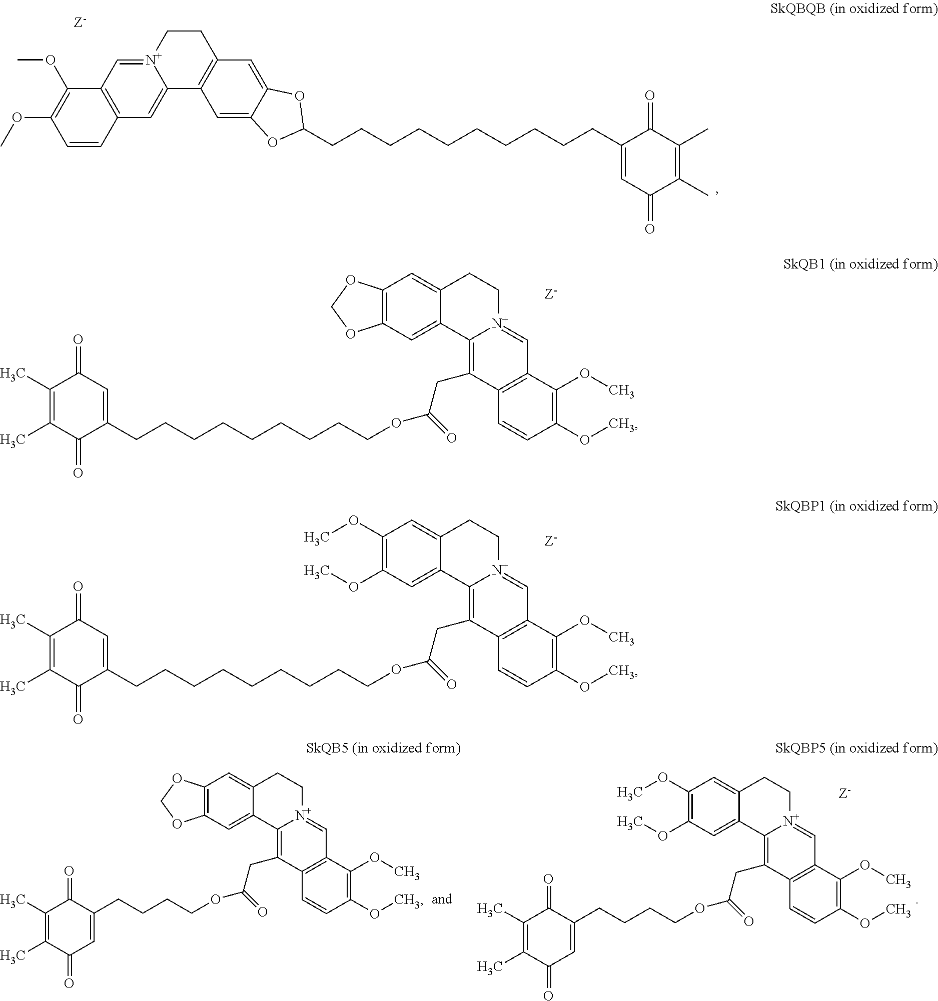

21. The formulation of any of the preceding claims, wherein the pharmaceutically acceptable anion Z⁻ comprises acetate, bromide, camsylate, chloride, formate, fumarate, maleate, mesylate, nitrate, oxalate, phosphate, dihydrogen phosphate dodecahydrate, dihydrogen phosphate dihydrate, sulfate, tartrate, thiocyanate, tosylate, adipate, caprate, caproate, caprylate, dodecylsulfate, glutarate, laurate, oleate, palmitate, sebacate, stearate, undecylenate, or a combination thereof.

22. The formulation of any of the preceding claims, wherein the SkQ optionally comprises a hydrate comprising one or more water molecules associated via a hydrogen bonding and/or an ionic bonding to the A, L, Sk⁺, and/or to Z⁻.

23. The formulation of any of the preceding claims, wherein the formulation is suitable for treating a subject at a dose of at least one drop or at least about 0.05 mL of the formulation to the eye and/or to one or more adnexa of the eye at least once every 24 hours.

24. The formulation of any of the preceding claims, wherein the formulation comprises one or more of the following: benzalkonium chloride, hypromellose, sodium chloride, sodium dihydrogen phosphate dihydrate, sodium dihydrogen phosphate dodecahydrate, and water.

25. The formulation of any of the preceding claims, wherein the Schirmer's test score is an unanesthetized Schirmer's test score.

26. The formulation of any of the preceding claims, wherein the one or more symptoms of dry eye disease comprise one or more symptoms reportable by the subject.

27. The formulation of any of the preceding claims, wherein the one or more signs and/or the one or more symptoms of dry eye disease arise from a progressive dry eye disease for longer than about 2 months.

28. The formulation of claim 27, wherein a damage to the central cornea is indicated by a grade of central corneal fluorescein staining of ≥1.

29. The formulation of any of the preceding claims, wherein the formulation comprises SkQ at a concentration of ≥1 μg/mL.

30. A kit for treating one or more signs and/or one or more symptoms of dry eye disease, the kit comprising:
a dispensing container comprising an eye-drop or eye-gel formulation comprising SkQ at a concentration of ≥0.5 μg/mL; and
instructions for use.

31. The kit of claim 28, wherein the eye-drop or eye-gel formulation comprises SkQ at a concentration of ≥1 μg/mL.

32. Use of the formulation of any of claims 1-29 or the kit of claim 30 or 31 to treat or prevent dry eye syndrome in a subject in need thereof.

33. The use of claim 32, wherein the subject's eye has a tear production of ≥7 mm measured by a Schirmer's test,
